(19) United States
(12) Patent Application Publication
(10) Pub. No.: US 2017/0237848 A1
(43) Pub. Date: Aug. 17, 2017

(54) SYSTEMS AND METHODS TO DETERMINE USER EMOTIONS AND MOODS BASED ON ACCELERATION DATA AND BIOMETRIC DATA

(71) Applicant: Lenovo (Singapore) Pte. Ltd., New Tech Park (SG)

(72) Inventors: Mark Charles Davis, Durham, NC (US); Richard Wayne Cheston, Pittsboro, NC (US); Howard Jeffrey Locker, Cary, NC (US); Robert A. Bowser, Cary, NC (US); Goran Hans Wibran, Cary, NC (US)

(21) Appl. No.: 15/583,127

(22) Filed: May 1, 2017

Related U.S. Application Data
(63) Continuation of application No. 14/132,451, filed on Dec. 18, 2013.

(51) Int. Cl. H04M 1/725; G06F 3/0482; G06F 3/0346; A61B 5/16; G06F 3/01

(52) U.S. Cl. CPC H04M 1/72569; H04M 1/72577; A61B 5/165; G06F 3/017; G06F 3/0346; G06F 3/012; G06F 3/0482

(57) ABSTRACT

In one aspect, a device includes an accelerometer, a processor and a memory accessible to the processor. The memory bears instructions executable by the processor to receive first data from a biometric sensor which communicates with the device, and receive second data from the accelerometer. The first data pertains to a biometric of a user and the second data pertains to acceleration of the device. The memory also bears instructions executable by the processor to determine one or more emotions of the user based at least partially on the first data and the second data, and determine whether to execute a function at the device at least partially based on the emotion and based on third data associated with a use context of the device.

### FIELD

The present application relates generally to determining emotions and moods of a user of a device.

### BACKGROUND

Interaction between users and their devices can be improved if the device were able to access data on the user's emotions and moods. Heretofore, there have not been provided adequate solutions for determining a user's mood or emotion with an acceptable degree of accuracy using a device.

### SUMMARY

Accordingly, in a first aspect a device includes an accelerometer, a processor and a memory accessible to the processor. The memory bears instructions executable by the processor to receive first data from a biometric sensor which communicates with the device, and receive second data from the accelerometer. The first data pertains to a biometric of a user and the second data pertains to acceleration of the device. The memory also bears instructions executable by the processor to determine one or more emotions of the user based at least partially on the first data and the second data, and determine whether to execute a function at the device at least partially based on the emotion and based on third data associated with a use context of the device.

The first data and second data may be received substantially in real time as it is respectively gathered by the biometric sensor and accelerometer, if desired. The use context may pertain to a current use of the device, and may be associated with a detected activity in which the user is engaged. In addition to or in lieu of the foregoing, the third data may include information from a use context history for the device.

In some embodiments, the third data may also include first global positioning system (GPS) coordinates for a current location of the device, and the instructions may be executable by the processor to determine whether to execute the function at least partially based on a determination that the first GPS coordinates are proximate to the same location as second GPS coordinates from the use context history. Furthermore, if desired the second GPS coordinates may be associated in the use context history with a detected activity in which the user has engaged, where the detected activity may at least in part establish the use context, and the instructions may be executable by the processor to determine whether to execute the function at least partially based on the detected activity.

In addition, in some embodiments the instructions may be executable by the processor to determine to execute the function at least partially based on the emotion and based on the third data, and then execute the function. The instructions may also be executable by the processor to determine to decline to execute the function at least partially based on the emotion and based on the third data.

Moreover, in some embodiments the instructions may be executable by the processor to determine the one or more emotions of the user based at least partially on the first data, the second data, and fourth data from a camera in communication with the device. The fourth data may be associated with an image of the user's face gathered by the camera, and the emotion may be determined at least in part by processing the fourth data using emotion recognition software.

Also in some embodiments, the second data may be determined to pertain to acceleration of the device beyond an acceleration threshold, and the instructions may be executable by the processor to determine the emotion of anger at least partially based on the second data.

In another aspect, a method includes receiving first data pertaining to at least one biometric of a user of a device, receiving second data pertaining to acceleration of the device, and determining one or more moods that correspond to both the first data and the second data.

In still another aspect, a device includes an accelerometer, at least one biometric sensor, a camera, a processor, and a memory accessible to the processor. The memory bears instructions executable by the processor to receive first data from the biometric sensor, and receive second data from the accelerometer. The first data pertains to a biometric of a user associated with the device, and the second data pertains to acceleration of the device. The memory also bears instructions executable by the processor to receive third data from the camera pertaining to an image of the user, and determine one or more emotions that correspond to the first data, the second data, and the third data.

The details of present principles, both as to their structure and operation, can best be understood in reference to the accompanying drawings, in which like reference numerals refer to like parts, and in which:

### BRIEF DESCRIPTION OF THE DRAWINGS

FIG. 1 is a block diagram of an example system in accordance with present principles;

FIGS. 2-4 are exemplary flowcharts of logic to be executed by a system in accordance with present principles;

FIGS. 5, 6, and 8 are exemplary data tables in accordance with present principles; and

FIG. 7 is an exemplary user interface (UI) presentable on the display of a system in accordance with present principles.

### DETAILED DESCRIPTION

This disclosure relates generally to device based user information. With respect to any computer systems discussed herein, a system may include server and client components, connected over a network such that data may be exchanged between the client and server components. The client components may include one or more computing devices including televisions (e.g. smart TVs, Internet-enabled TVs), computers such as desktops, laptops and tablet computers, and other mobile devices including smart phones. These client devices may employ, as non-limiting examples, operating systems from Apple, Google, or Microsoft. A Unix operating system may be used. These operating systems can execute one or more browsers such as a browser made by Microsoft or Google or Mozilla or other browser program that can access web applications hosted by the Internet servers over a network such as the Internet, a local intranet, or a virtual private network.

As used herein, instructions refer to computer-implemented steps for processing information in the system. Instructions can be implemented in software, firmware or hardware; hence, illustrative components, blocks, modules, circuits, and steps are set forth in terms of their functionality.

A processor may be any conventional general purpose single- or multi-chip processor that can execute logic by means of various lines such as address lines, data lines, and control lines and registers and shift registers. Moreover, any logical blocks, modules, and circuits described herein can be implemented or performed, in addition to a general purpose processor, in or by a digital signal processor (DSP), a field programmable gate array (FPGA) or other programmable logic device such as an application specific integrated circuit (ASIC), discrete gate or transistor logic, discrete hardware components, or any combination thereof designed to perform the functions described herein. A processor can be implemented by a controller or state machine or a combination of computing devices.

Any software and/or applications described by way of flow charts and/or user interfaces herein can include various sub-routines, procedures, etc. It is to be understood that logic divulged as being executed by e.g. a module can be redistributed to other software modules and/or combined together in a single module and/or made available in a shareable library.

Logic when implemented in software, can be written in an appropriate language such as but not limited to C# or C++, and can be stored on or transmitted through a computer-readable storage medium (e.g. that may not be a carrier wave) such as a random access memory (RAM), read-only memory (ROM), electrically erasable programmable read-only memory (EEPROM), compact disk read-only memory (CD-ROM) or other optical disk storage such as digital versatile disc (DVD), magnetic disk storage or other magnetic storage devices including removable thumb drives, etc. A connection may establish a computer-readable medium. Such connections can include, as examples, hard-wired cables including fiber optics and coaxial wires and twisted pair wires. Such connections may include wireless communication connections including infrared and radio.

In an example, a processor can access information over its input lines from data storage, such as the computer readable storage medium, and/or the processor can access information wirelessly from an Internet server by activating a wireless transceiver to send and receive data. Data typically is converted from analog signals to digital by circuitry between the antenna and the registers of the processor when being received and from digital to analog when being transmitted. The processor then processes the data through its shift registers to output calculated data on output lines, for presentation of the calculated data on the device.

Components included in one embodiment can be used in other embodiments in any appropriate combination. For example, any of the various components described herein and/or depicted in the Figures may be combined, interchanged or excluded from other embodiments.

“A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together, etc.

The term “circuit” or “circuitry” is used in the summary, description, and/or claims. As is well known in the art, the term “circuitry” includes all levels of available integration, e.g., from discrete logic circuits to the highest level of circuit integration such as VLSI, and includes programmable logic components programmed to perform the functions of an embodiment as well as general-purpose or special-purpose processors programmed with instructions to perform those functions.

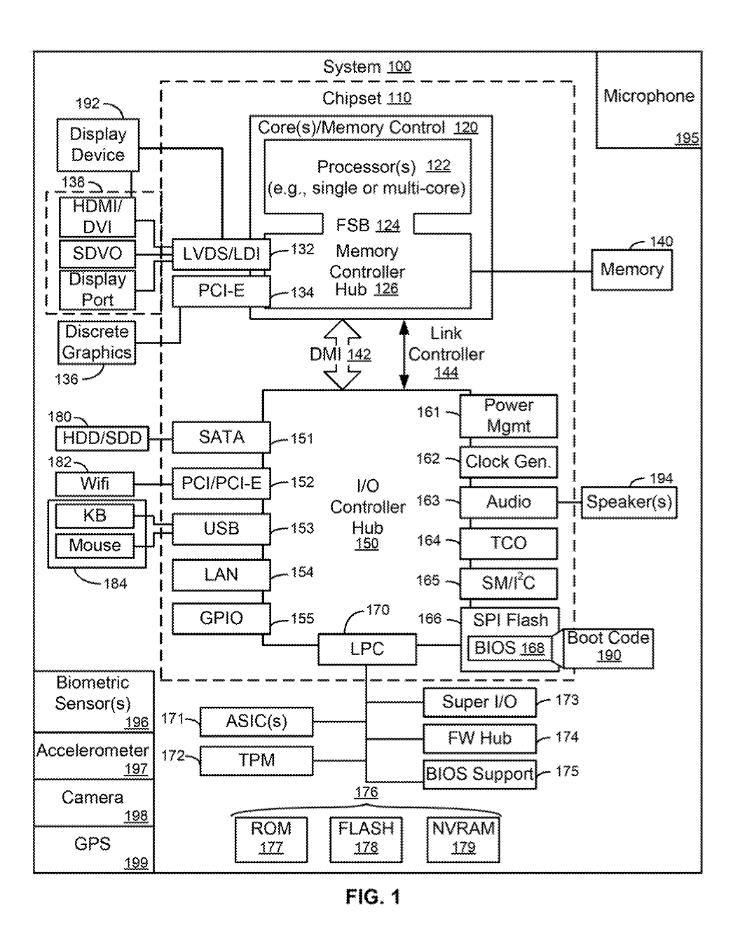

Now specifically in reference to FIG. 1, it shows an exemplary block diagram of an information handling system and/or computer system 100 such as e.g. an Internet enabled, computerized telephone (e.g. a smart phone), a tablet computer, a notebook or desktop computer, an Internet enabled computerized wearable device such as a smart watch, a computerized television (TV) such as a smart TV, etc. Thus, in some embodiments the system 100 may be a desktop computer system, such as one of the ThinkCentre® or ThinkPad® series of personal computers sold by Lenovo (US) Inc. of Morrisville, N.C., or a workstation computer, such as the ThinkStation®, which are sold by Lenovo (US) Inc. of Morrisville, N.C.; however, as apparent from the description herein, a client device, a server or other machine in accordance with present principles may include other features or only some of the features of the system 100.

As shown in FIG. 1, the system 100 includes a so-called chipset 110. A chipset refers to a group of integrated circuits, or chips, that are designed to work together. Chipsets are usually marketed as a single product (e.g., consider chipsets marketed under the brands INTEL®, AMD®, etc.).

In the example of FIG. 1, the chipset 110 has a particular architecture, which may vary to some extent depending on brand or manufacturer. The architecture of the chipset 110 includes a core and memory control group 120 and an I/O controller hub 150 that exchange information (e.g., data, signals, commands, etc.) via, for example, a direct management interface or direct media interface (DMI) 142 or a link controller 144. In the example of FIG. 1, the DMI 142 is a chip-to-chip interface (sometimes referred to as being a link between a “northbridge” and a “southbridge”).

The core and memory control group 120 include one or more processors 122 (e.g., single core or multi-core, etc.) and a memory controller hub 126 that exchange information via a front side bus (FSB) 124. As described herein, various components of the core and memory control group 120 may be integrated onto a single processor die, for example, to make a chip that supplants the conventional “northbridge” style architecture.

The memory controller hub 126 interfaces with memory 140. For example, the memory controller hub 126 may provide support for DDR SDRAM memory (e.g., DDR, DDR2, DDR3, etc.). In general, the memory 140 is a type of random-access memory (RAM). It is often referred to as “system memory.”

The memory controller hub 126 further includes a low-voltage differential signaling interface (LVDS) 132. The LVDS 132 may be a so-called LVDS Display Interface (LDI) for support of a display device 192 (e.g., a CRT, a flat panel, a projector, a touch-enabled display, etc.). A block 138 includes some examples of technologies that may be supported via the LVDS interface 132 (e.g., serial digital video, HDMI/DVI, display port). The memory controller hub 126 also includes one or more PCI-express interfaces (PCI-E) 134, for example, for support of discrete graphics 136. Discrete graphics using a PCI-E interface has become an alternative approach to an accelerated graphics port (AGP). For example, the memory controller hub 126 may include a 16-lane (x16) PCI-E port for an external PCI-E-based graphics card (including e.g. one of more GPUs). An exemplary system may include AGP or PCI-E for support of graphics.

The I/O hub controller 150 includes a variety of interfaces. The example of FIG. 1 includes a SATA interface 151, one or more PCI-E interfaces 152 (optionally one or more legacy PCI interfaces), one or more USB interfaces 153, a LAN interface 154 (more generally a network interface for communication over at least one network such as the Internet, a WAN, a LAN, etc. under direction of the processor(s) 122), a general purpose I/O interface (GPIO) 155, a low-pin count (LPC) interface 170, a power management interface 161, a clock generator interface 162, an audio interface 163 (e.g., for speakers 194 to output audio), a total cost of operation (TCO) interface 164, a system management bus interface (e.g., a multi-master serial computer bus interface) 165, and a serial peripheral flash memory/controller interface (SPI Flash) 166, which, in the example of FIG. 1, includes BIOS 168 and boot code 190. With respect to network connections, the I/O hub controller 150 may include integrated gigabit Ethernet controller lines multiplexed with a PCI-E interface port. Other network features may operate independent of a PCI-E interface.

The interfaces of the I/O hub controller 150 provide for communication with various devices, networks, etc. For example, the SATA interface 151 provides for reading, writing or reading and writing information on one or more drives 180 such as HDDs, SDDs or a combination thereof, but in any case the drives 180 are understood to be e.g. tangible computer readable storage mediums that may not be carrier waves. The I/O hub controller 150 may also include an advanced host controller interface (AHCI) to support one or more drives 180. The PCI-E interface 152 allows for wireless connections 182 to devices, networks, etc. The USB interface 153 provides for input devices 184 such as keyboards (KB), mice and various other devices (e.g., cameras, phones, storage, media players, etc.).

In the example of FIG. 1, the LPC interface 170 provides for use of one or more ASICs 171, a trusted platform module (TPM) 172, a super I/O 173, a firmware hub 174, BIOS support 175 as well as various types of memory 176 such as ROM 177, Flash 178, and non-volatile RAM (NVRAM) 179. With respect to the TPM 172, this module may be in the form of a chip that can be used to authenticate software and hardware devices. For example, a TPM may be capable of performing platform authentication and may be used to verify that a system seeking access is the expected system.

The system 100, upon power on, may be configured to execute boot code 190 for the BIOS 168, as stored within the SPI Flash 166, and thereafter processes data under the control of one or more operating systems and application software (e.g., stored in system memory 140). An operating system may be stored in any of a variety of locations and accessed, for example, according to instructions of the BIOS 168.

In addition to the foregoing, the system 100 is understood to include an audio receiver/microphone 195 in communication with the processor 122 and providing input thereto based on e.g. a user providing audible input to the microphone 195 in accordance with present principles. One or more biometric sensors 196 are also shown that are in communication with the processor 122 and provide input thereto, such as e.g. heart rate sensors and/or heart monitors, blood pressure sensors, iris and/or retina detectors, oxygen sensors (e.g. blood oxygen sensors), glucose and/or blood sugar sensors, pedometers and/or speed sensors, body temperature sensors, etc. Furthermore, the system 100 may include one or more accelerometers 197 or other motion sensors such as e.g. gesture sensors (e.g. for sensing gestures in free space associated by the device with moods and/or emotions in accordance with present principles) that are in communication with the processor 122 and provide input thereto.

A camera 198 is also shown, which is in communication with and provides input to the processor 122. The camera 198 may be, e.g., a thermal imaging camera, a digital camera such as a webcam, and/or a camera integrated into the system 100 and controllable by the processor 122 to gather pictures/images and/or video in accordance with present principles (e.g. to gather one or more images of a user's face to apply emotion recognition software to the image(s) in accordance with present principles). In addition, a GPS transceiver 199 is shown that is configured to e.g. receive geographic position information from at least one satellite and provide the information to the processor 122. However, it is to be understood that another suitable position receiver other than a GPS receiver may be used in accordance with present principles to e.g. determine the location of the system 100.

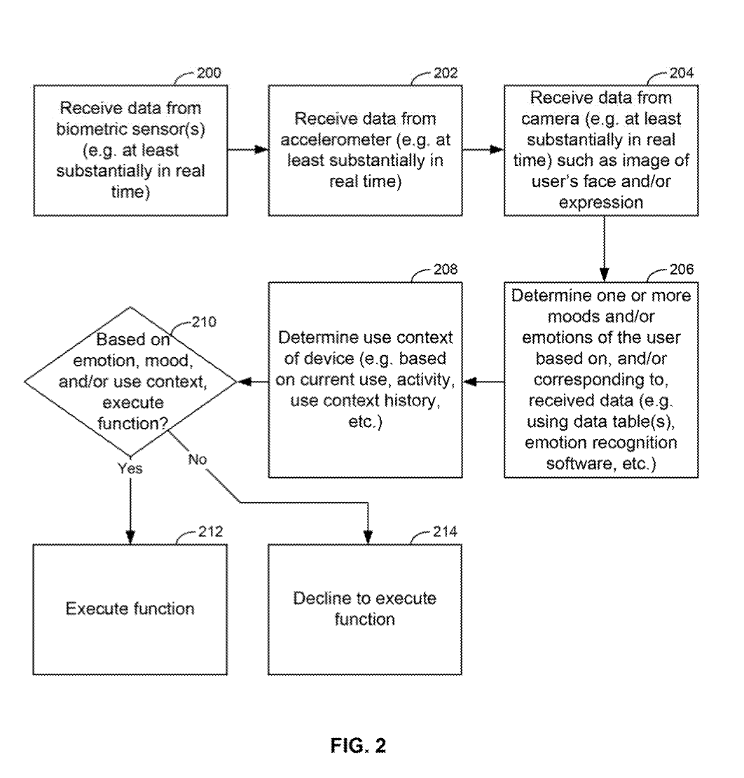

Before moving on to FIG. 2, it is to be understood that an exemplary client device or other machine/computer may include fewer or more features than shown on the system 100 of FIG. 1. In any case, it is to be understood at least based on the foregoing that the system 100 is configured to undertake present principles.

Now in reference to FIG. 2, an example flowchart of logic to be executed by a device such as the system 100 described above in accordance with present principles is shown. Beginning at block 200, the logic receives data from a biometric sensor pertaining to a biometric of a user of the system 100 e.g. in real time or substantially in real time as the data is gathered by the biometric sensor. Then at block 202 the logic receives data from an accelerometer pertaining to acceleration of the device e.g. in real time or substantially in real time as the data is gathered by the accelerometer. Thereafter at block 204, the logic receives data from a camera (e.g. such as one on the device) in real time or substantially in real time as the data is gathered by the camera. The data from the camera may be e.g. an image(s) of the user such as the user's face, and/or may pertain to a user's facial expression.

After block 204, the logic proceeds to block 206 where the logic determines one or more emotions and/or moods of the user in accordance with present principles, such as e.g. based at least partially on and/or corresponding to the data from the biometric sensor, and/or the data from the accelerometer, and/or the data from the camera. The determination made at block 206 may be made by e.g. parsing a data table correlating biometric output of a user with one or more emotions and/or moods, and/or parsing a data table correlating acceleration with one or more emotions and/or moods, to thus identify the one or more emotions or moods. Exemplary data tables will be discussed further below. However, note that still other ways of determining one or more emotions and/or moods corresponding to and/or based on the data may be used, such as e.g. executing and/or applying emotion recognition software to the data (e.g., applying the software to an image of the user's face to determine one or more emotions the user is expressing with his or her face).

Still in reference to FIG. 2, after block 206 the logic proceeds to block 208 where the logic determines one or more use contexts of the device e.g. based on a current use of the device (e.g. an application and/or function on the device with which the user is engaged, use tracking software on the device, etc.), based on an activity in which the user is engaged as sensed by and/or determined by the device, and/or based on a use context history in accordance with present principles, etc. Thereafter, the logic proceeds to decision diamond 210 where the logic determines whether to execute a function at or on the device at least partially based on the emotion and/or mood, and/or the use context. This may be done by e.g. parsing a data table correlating emotions with functions, and/or use contexts with functions, to thus identify the functions.

Continuing in reference to diamond 210, should an affirmative determination be made thereat, the logic proceeds to block 212 where the logic executes the function. However, a negative determination at diamond 210 causes the logic to move instead to block 214 where the logic declines to execute the function.

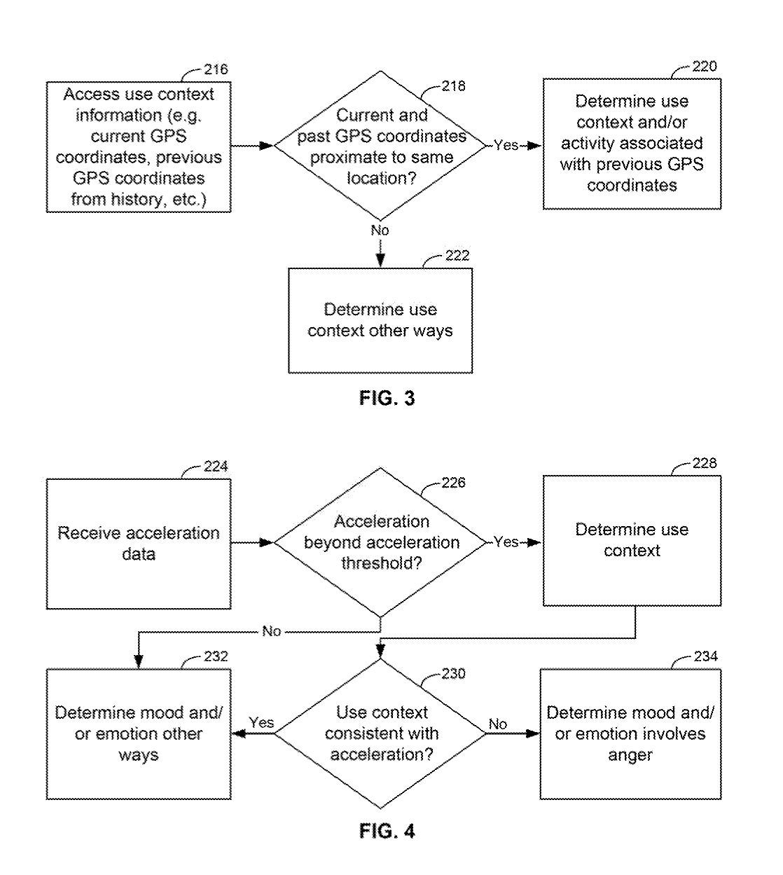

Moving from FIG. 2 to FIG. 3, it shows exemplary logic for determining a use context in accordance with present principles, it being thus understood that the logic of FIG. 3 (and/or also FIG. 4) maybe used in conjunction with (e.g. incorporated with) the logic of FIG. 2. Beginning at block 216, the logic accesses information for determining the use context, which in the present instance includes e.g. GPS coordinates for a current location of the device as received from a GPS transceiver of the device, and also includes previous GPS coordinates indicated in a location history and/or use context history of the device. The logic then proceeds to decision diamond 218 where the logic determines whether the current GPS coordinates and GPS coordinates from the history or histories are proximate to each other and/or at the same location (e.g. a particular location such as a tennis court, a concert venue, an office or personal residence, etc.). The coordinates may be determined to be proximate e.g. based on being within a threshold distance of each other and/or the same location, where the threshold distance may be predefined and/or user defined (e.g. using a settings user interface for configuring various functions in accordance with present principles).

An affirmative determination at diamond 218 causes the logic to proceed to block 220 where the logic determines an (e.g. current) use context and/or particular activity in which the user is engaging based on the current GPS coordinates being proximate to the same location as GPS coordinates indicated in a data table and/or history which are associated with the use context and/or activity. However, a negative determination at diamond 218 instead causes the logic to move to block 222 where the logic may determine a use context and/or activity in other ways as disclosed herein.

Continuing the detailed description in reference to FIG. 4, it shows exemplary logic for determining a user's mood and/or emotions based on at least partially on whether an acceleration threshold has been reached in accordance with present principles. The logic begins at block 224 where the logic receives acceleration data e.g. from an accelerometer on a device such as the system 100. The logic then proceeds to decision diamond 226 where the logic determines based on the acceleration data whether acceleration of the device has met and/or exceeded an acceleration threshold (e.g. is greater than a predetermined amount of acceleration establishing the threshold as e.g. defined by a user). The logic may do so at diamond 226 by e.g. taking an amount of acceleration from the data received at block 224 and comparing it to the threshold to determine whether the amount of acceleration from the data is at and/or above the threshold amount.

An affirmative determination at diamond 226 causes the logic to proceed to block 228 where the logic determines a use context in accordance with present principles e.g. at least partially based on the acceleration being at or past the acceleration threshold (e.g. based on the acceleration amount above the threshold amount being indicated in a data table as correlating to a use context such as e.g. exercising or slamming the device down on a desk). However, a negative determination at diamond 226 causes the logic to instead proceed to block 232, which will be described shortly. But before doing so, reference is made to decision diamond 230, which is arrived at from block 228. At diamond 230, the logic determines whether the use context determined at block 228 is consistent with the acceleration indicated in the acceleration data. For instance, if the use context and/or activity was wearing the device while playing tennis to track tennis-related movements and biometric output of the user, relatively rapid acceleration would be consistent with and/or correlated with playing tennis (e.g. as indicated in a data table). Thus, an affirmative determination at diamond 230 causes the logic to proceed to block 232 where the logic determines the user's mood and/or emotions in other ways since e.g. the acceleration even though beyond the acceleration threshold is consistent with a particular physical activity with which relatively rapid acceleration is to be expected. However, a negative determination at diamond 230 instead causes the logic to proceed to block 234 where the logic determines the user's mood and/or emotions to include anger since e.g. the device has not determined a use context consistent with the acceleration that was detected.

For instance, if the user were in the user's office rather than on the tennis court, and the device detects acceleration beyond the acceleration threshold, it may be determined that the user is not playing tennis but instead engaging in something else causing the relatively rapid acceleration that was detected and hence may be angry (e.g. the acceleration being generated by the user slamming the device down on the user's desk).

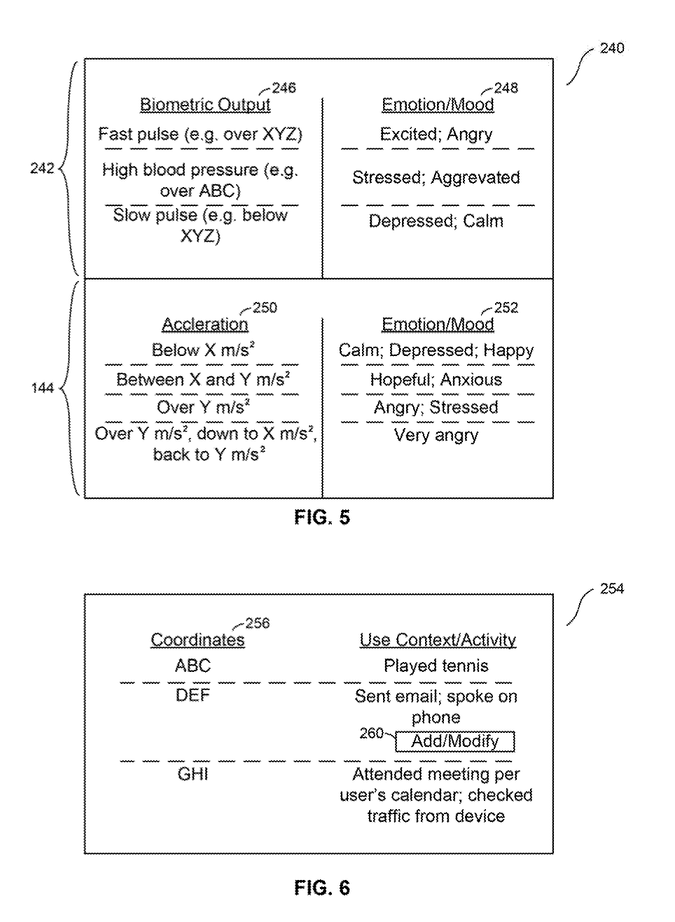

Turning to FIG. 5, it shows an exemplary data table 240 for correlating biometric output with one or more emotions and/or moods, and/or for correlating acceleration to one or more emotions and/or moods. Accordingly, it is to be understood that such a data table as shown in FIG. 5 may be used in accordance with the principles set forth herein to determine the user's emotion(s) and/or mood(s) (e.g. used while a device undertakes the logic of FIG. 2). The table 240 thus includes a first section 242 correlating biometric output with one or more emotions and/or moods, and a section 244 correlating acceleration to one or more emotions and/or moods. However, it is to be understood that the respective information and/or data in the sections 242 and 244 may be included in respective separate data tables, if desired.

Regardless, the first section 242 includes a first column 246 pertaining to types and/or amounts of biometric output, and a second column 248 pertaining to moods and/or emotions associated with the respective types and/or amounts of biometric output. Thus, for instance, a device in accordance with present principles may detect biometric output for a user's pulse and determine that it is over the pulse threshold amount of XYZ, and then parse the data table 240 to locate a biometric output entry for a pulse being over the pulse threshold amount of XYZ to then determine that the emotions associated therewith in the data table 240 are excitement and anger. As another example, after receiving biometric output for blood pressure that is over a threshold amount of ABC, the logic may access and parse the data table 240 to locate a biometric output entry for blood pressure being over the threshold amount ABC to then determine the emotions associated therewith in the data table 240, which in this case are e.g. stress and aggravation.

Describing the second section 244, it includes a first column 250 pertaining to types (e.g. linear and non-linear) acceleration and/or amounts of acceleration, and a second column 252 pertaining to moods and/or emotions associated with the respective types and/or amounts of acceleration. For instance, a device in accordance with present principles may detect acceleration but below a threshold acceleration of X meters per second squared, and then parse the data table 240 to locate an acceleration entry for acceleration below X meters per second squared to identify at least emotion associated therewith in the data table 240, which in the present exemplary instance is one or more of being calm, depressed, or happy. As another example, the device may detect acceleration above a threshold acceleration of Y meters per second squared, and based on parsing the data table 240 in accordance with present principles identify emotions and/or moods associated with acceleration above the threshold Y meters per second squared as including being angry and/or stressed. As a third example, acceleration detected over Y meters per second squared, then acceleration detected at around X meters per second squared, and then acceleration again detected as increasing back to Y meters per second squared may be determined to be associated based on the data table 240 with the emotion of being very angry (e.g. should the user be hectically moving the device around in disgust).

Providing an example of using both biometric data and acceleration to identify at least one common emotion or mood associated with both (e.g. as correlated in a data table such as the table 240), the device may receive biometric data for the user's pulse indicative of the user's pulse being over the threshold XYZ, and may also receive acceleration data indicating an acceleration of the device over Y meters per second squared. The device may then, using the data table 240, determine that the emotion of anger is associated with both a pulse above XYZ and acceleration over Y meters per second squared, and hence determine based on the biometric and acceleration data that the user is experiencing the emotion of anger (e.g. after also determining based on use context that the user is not e.g. at a tennis court playing tennis, which may also cause the user's pulse to increase past XYZ and acceleration to be detected over Y meters per second squared). Note that only anger has been identified since e.g. the emotion of being excited is not correlated to acceleration over Y meters per second squared and the emotion of being excited is not correlated to a pulse above XYZ.

Continuing the detailed description in reference to FIG. 6, it shows a data table 254 for correlating GPS coordinates with a use context and/or activity, which in some embodiments may also at least in part establish a use context history having a representation presentable to a user (e.g. on a display of the device) in accordance with present principles. The table 254 includes a first column 256 listing entries of GPS coordinates for locations at which the device was previously located, along with respective entries for use contexts and/or activities associated therewith in a second column 258. It is to be understood that the data table 254 may be generated by the device by e.g. determining GPS coordinates for the device at a particular location, and then determining a use context and/or activity for the location e.g. based on user input indicative of an activity, based on electronic calendar information for the user, based on device functions controlled by and/or engaged in by the user at the location, based on location information such as a particular establishment indicated on an electronic map accessible to the device, etc., to thus associate the coordinates with the use context and/or activity, and then enter and/or establish the correlation in the data table 254. Thus, e.g. based on calendar information indicating a time at which the user was to play tennis, and based on the device being at coordinates ABC at that time, the device may correlate the coordinates with the activity of playing tennis.

In any case, it is to be understood that when e.g. comparing current GPS coordinates to coordinates in a table such as the table 254 as described herein, the current GPS coordinates may be matched to an entry in column 256 to thereby determine a use context or activity associated with the entry. For example, supposed a device is currently at a location with GPS coordinates GHI. The device may access the table 254, match the coordinates GHI as being at least proximate to a previous location indicated in the table 254 (in this case the device is at the same location corresponding to coordinates GHI as during a previous instance), and thus determine at least one use context and/or activity associated with the coordinates GHI based on the coordinates GHI being correlated in the data table with the user attending a meeting (e.g. as was indicated on the user's calendar), and also checking traffic congestion from the device at the location.

Still in reference to FIG. 6, note that when e.g. the data table 254 is presented in a visual representation on the device, a selector element 260 may be presented for at least one and optionally all of the entries in either or both columns 256 and 258 to modify the entry (provide manual input to add, delete, or modify the entry). For instance, the user may wish that a particular activity be associated with particular coordinates, and then provide input to the device to cause the data table to correlate the user's indicated activity with the associated coordinates.

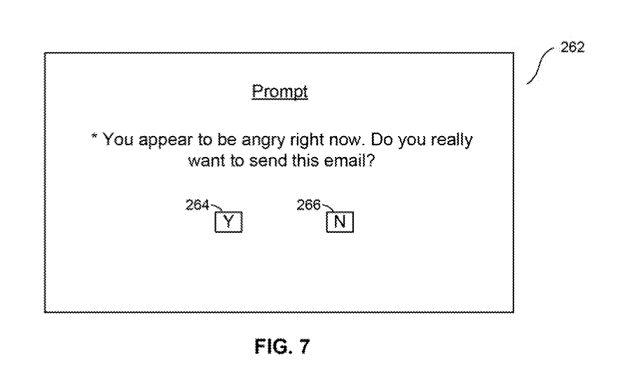

Moving on, reference is now made to FIG. 7. It shows an exemplary user interface 262 that may be e.g. a prompt presented on a device such as the system 100 for whether the user desires the device to execute a function for which the user has already provided a command and/or input, such as e.g. sending an email. For instance, the prompt may indicate that the device has determined the user as being angry (e.g. based on executing the logic set forth herein), and hence may present the prompt 262 after a user completes an email while angry and provides input to the device to send the email (to thus prompt the user to confirm that they wish to send the email despite being angry). Accordingly, it is to be understood that in this example, while the user has already provided input to send the email, based on the device determining that the user is angry the device has not actually sent the email yet but has presented the prompt 262 to confirm the user wishes to send it. Thus, a yes selector element 264 is presented and is selectable to automatically without further user input send the email, while a no selector element 266 is also shown which is selectable to automatically without further user input cause the device to decline to process the user's previous input to send the email and hence decline to send the email.

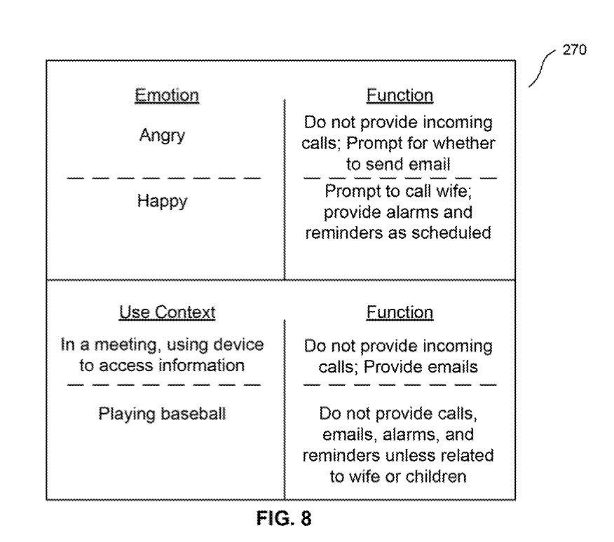

Before moving on to FIG. 8, it is to be understood that a settings UI may be presented on a device in accordance with present principles to configure one or more of the features, elements, functions, etc. disclosed herein. Thus, for instance, a user may access a settings UI presentable on a display of the device and configure settings for prompts such as the prompt 262. E.g., the settings UI may enable the user to turn on or off the prompt feature requesting confirmation before executing a function (e.g. when the user is in a particular emotional state). Furthermore, the user may even e.g. provide input using the settings UI for particular emotions that, if detected by the device, may cause a prompt like the prompt 262 to be presented while other emotions may not and instead simply cause the device to execute the function responsive to the user's input to do so.

Now in reference to FIG. 8, it shows an exemplary data table 270 correlating emotions with functions to be executed by the device, and/or use contexts with functions to be executed by the device, in accordance with present principles. For instance, the table 270 may correlate the emotion of angry with declining to provide incoming calls to the user, and to provide a confirmation prompt to the user after the user provides input to send an email when the user is angry. If the user is determined to be happy, the device may access the data table to determine functions correlated therewith, such as e.g. presenting a prompt to call the user's wife, and to provide alarms and reminders programmed into the device as scheduled.

As another example, if a use context is that a user is in a meeting and is using the device to access information (e.g. over the Internet), the function correlated therewith may be to decline to provide incoming calls but to nonetheless provide emails and/or email notifications to the user while in the meeting.

Without reference to any particular figure, it is to be understood that although e.g. an application for undertaking present principles may be vended with a device such as the system 100, it is to be understood that present principles apply in instances where such an application is e.g. downloaded from a server to a device over a network such as the Internet.

Also without reference to any particular figure, it is to be understood that the histories, data tables, etc. disclosed herein may be stored locally on the device undertaking present principles (e.g. on a computer readable storage medium of the device), and or stored remotely such as at a server and/or in cloud storage.

Furthermore, gestures in free space may also be detected by a device in accordance with present principles, may be correlated with one or more moods and/or emotions in accordance with present principles (e.g. in a data table), and thus may be used to make determinations in accordance with present principles. For instance, a gesture recognized by the device (e.g. based on received gesture data from a gesture sensor being applied to gesture recognition software to identify the gesture) may be correlated in a data table as being associated with happiness, and the device may take one or more actions accordingly. The same applies to voice input received through a microphone, mutatis mutandis.

Still without reference to any particular figure, it is to be understood that present principles may apply e.g. when acceleration is detected in more than one dimension as well. E.g. acceleration above a first threshold amount in one dimension and above a second threshold amount in another dimension may be indicative of a particular emotion of a user, while acceleration only in one dimension and/or above only the first threshold may be indicative of another emotion.

Furthermore, it is to be understood that although GPS transceivers and GPS coordinates have been disclosed above in accordance with present principles, it is to be understood that still other ways of determining, identifying, comparing, etc. locations may be used in accordance with present principles. For instance, (e.g. indoor) location may be determined using triangulation techniques that leverage wireless LANs and/or Bluetooth proximity profiles.

Before concluding, also note that the tables described herein may be changed and updated (e.g. over time) depending on results of previous logic determinations, user input, user feedback (e.g. if the user indicates using input to a UI that the mood and/or emotion that was determined was incorrect), etc. For instance, a device in accordance with present principles may present a prompt after making a determination of one or more moods and/or emotions that indicates the mood and/or emotion that has been determined and requests verification that the determined mood and/or emotion is correct and/or corresponds to an actual mood and/or emotion being experienced by the user. E.g., the prompt may indicate, “I think you are angry. Is this correct?” and then provide yes and no selector elements for providing input regarding whether or not the device's determination corresponds to the user's actual emotional state. One or more portions of the data tables may then up updated such as e.g. acceleration being at the detected level not necessarily (e.g. any longer) corresponding to the emotion that was previously correlated therewith in the table, and hence possibly even removing the determined emotion from the table entry to thus no longer be correlated with the detected acceleration level.

It may now be appreciated that biometric information and device acceleration data may be used to determine a user's mood and/or emotions, which may itself be used to determine an action to take or not take based on the mood or emotion. Acceleration data may indicate activity levels and emotional states of the user. Furthermore, the acceleration data may be uneven acceleration (e.g. non-linear) and/or (e.g. relatively) even acceleration (e.g. linear), and such linear and non-linear acceleration may be indicative of different emotions. Present principles may thus be undertaken by a wearable device such a smart watch having one or more health monitors, activity sensors, etc. The types of biometrics that may be used in accordance with present principles include but are not limited to e.g. temperature, pulse, heart rate, etc.

It may also be appreciated that present principles provide systems and methods for a device to determine the activity level of a user and the emotional state of the user using one or both of at least acceleration data and biometric data. In some exemplary embodiments, significant periodic acceleration may be determined to indicate that the user is walking briskly (e.g. such as through an airport), and that it is thus not a good time to remind the user about a meeting that is scheduled to occur per the user's calendar in fifteen minutes. However, e.g. a meeting schedule to occur in two minutes may be indicated in a notification with a relatively high volume that may increase as the scheduled event continues to approach in time. Intermittent relatively very high acceleration may be indicative of anger in some instances, while in other instance may simply be indicative of the user playing tennis. Historical analysis and context analysis may be undertaken by a device in accordance with present principles to disambiguate e.g. the anger or tennis. The device's responsiveness to a certain set of parameters may then be adjusted to the user's emotional state such as putting up an e.g. “Are you sure?” notification before sending an email.

While the particular SYSTEMS AND METHODS TO DETERMINE USER EMOTIONS AND MOODS BASED ON ACCELERATION DATA AND BIOMETRIC DATA is herein shown and described in detail, it is to be understood that the subject matter which is encompassed by the present application is limited only by the claims.

What is claimed is:

1. A device, comprising:
a processor;
at least one gesture sensor accessible to the processor; and
storage accessible to the processor and bearing instructions executable by the processor to:
receive data from the gesture sensor;
identify, based on the data, a gesture performed by a user;
identify, based on the identification of the gesture, at least one emotion of the user;
determine, based on the identification of the at least one emotion of the user, whether to execute a function at the device;
responsive to a determination to execute the function at the device, execute the function at the device; and
responsive to a determination to not execute the function at the device, decline to execute the function at the device.

2. The device of claim 1, wherein the at least one gesture sensor comprises an accelerometer.

3. The device of claim 1, wherein the at least one gesture sensor comprises a camera.

4. The device of claim 1, wherein the instructions are executable by the processor to:
identify at least one emotion of the user at least partially based on identification of the user as gesturing a particular predefined gesture already associated with the identified at least one emotion.

5. The device of claim 4, wherein the particular predefined gesture is already associated with the identified at least one emotion in a database accessible to the processor that correlates particular gestures with particular emotions.

6. The device of claim 1, wherein the instructions are executable by the processor to:
identify the gesture based at least in part on execution of gesture recognition software to process the data.

7. The device of claim 1, wherein the gesture pertains to a facial expression of the user identified based on the data.

8. The device of claim 1, comprising a display, and wherein the instructions are executable by the processor to:
present a user interface (UI) on the display, the UI comprising a selector element at which presentation of prompts is enableable, the prompts being to confirm the user desires the device to execute functions for which user input has been provided despite a particular gesture being identified by the device.

9. The device of claim 1, wherein the at least one emotion that is identified comprises an emotion of angry, wherein the function is to provide an incoming telephone call at the device, and wherein the instructions are executable by the processor to:
determine, based on the identification of the emotion of angry, to decline to provide the telephone call at the device; and
decline to provide the telephone call at the device.

10. The device of claim 1, wherein the at least one emotion that is identified comprises an emotion of happy, wherein the function is to provide a prompt to call another person, and wherein the instructions are executable by the processor to:
determine, based on the identification of the emotion of happy, to provide the prompt at the device; and
provide the prompt at the device based on the determination to provide the prompt at the device.

11. The device of claim 1, wherein the function is to provide a notification at the device, and wherein the instructions are executable by the processor to:
determine, based on the identification of the at least one emotion of the user, whether to provide the notification at the device;
responsive to a determination to provide the notification at the device, provide the notification at the device; and
responsive to a determination to not provide the notification at the device, decline to provide the notification at the device.

12. A method, comprising:
receiving, at a device, data from at least one gesture sensor;
identifying, based on the data, a gesture performed by a user;
identifying, based on the identifying of the gesture, at least one emotion of the user;
determining, based on the identifying of the at least one emotion of the user, whether to execute a function at the device;
executing, responsive to determining to execute the function at the device, the function at the device; and
not executing, responsive to a determination to not execute the function at the device, the function at the device.

13. The method of claim 12, wherein the at least one gesture sensor comprises at least one of an accelerometer and a camera.

14. The method of claim 12, comprising:
identifying at least one emotion of the user at least partially based on identifying the user as gesturing a particular predefined gesture previously associated with the identified at least one emotion.

15. The method of claim 14, comprising:
accessing a database to identify the user as gesturing the particular predefined gesture previously associated with the identified at least one emotion.

16. The method of claim 12, comprising:
identifying the gesture based at least in part on execution of gesture recognition software to process the data.

17. The method of claim 12, wherein the gesture pertains to a facial expression of the user identified based on the data.

18. The method of claim 12, comprising:
presenting a user interface (UI) on a display accessible to the device, the UI comprising a selector element at which presentation of prompts is enableable, the prompts being to confirm the user desires the device to execute functions for which user input has been provided despite a particular gesture being identified by the device.

19. An apparatus, comprising:
a first processor;
a network adapter; and
storage bearing instructions executable by a second processor of a device for:
receiving data from a gesture sensor;
identifying, based on the data, a gesture performed by a user;
identifying, based on the identifying of the gesture, at least one emotion of the user;
determining, based on the identifying of the at least one emotion of the user, whether to execute a function at the device;
executing, responsive to determining to execute the function at the device, the function at the device; and
declining, responsive to determining to not execute the function at the device, to execute the function at the device;
wherein the first processor transfers the instructions to the device over a network via the network adapter.

20. The apparatus of claim 19, wherein the instructions are executable by the second processor for:
presenting a user interface (UI) on a display accessible to the device, the UI comprising a selector element at which presentation of prompts is enableable, the prompts being to confirm the user desires the device to execute functions for which user input has been provided despite a particular gesture being identified by the device.
